SEQUENCE LISTING (1 sequence)

SEQ ID NO: 1 - RNA, 234 nt, Q-beta bacteriophage
ggggacccccccggaagggggggacgaggugcgggcaccucguacgggaguucgaccgugacgcucuagagaucuagagcacgggcuagcgcuuucgcgcucucccaggugacgccucgugaagaggcgcgaccuucgugcguuucggugacgcacgagaaccgccacgcugcuucgcagcguggcuccuucgcgcagcccgcugcgcgaggugacccccgaagggggguuccc

(19) United States
(12) Patent Application Publication
(10) Pub. No.: US 2005/0272081 A1
(43) Pub. Date: Dec. 8, 2005

(54) Methods, compositions and apparatus for making nucleic acid molecules having a selected affinity to a target molecule

(75) Inventor: Abraham Grossman, Pleasantville, NY (US)

Correspondence Address: DAVIDSON, DAVIDSON & KAPPEL, LLC, 485 SEVENTH AVENUE, 14TH FLOOR, NEW YORK, NY, 10018, US

(73) Assignee: Q-RNA, Inc., New York, NY (US)

(21) Appl. No.: 11/135,673

(22) Filed: May 24, 2005

Related U.S. Application Data
(63) Continuation of application No. 09/752,231, filed on Dec. 29, 2000.
Application No. 09/752,231 is a continuation-in-part of application No. PCT/US99/15030, filed on Jul. 1, 1999.
(60) Provisional application No. 60/091,578, filed on Jul. 2, 1998.

(51) Int. Cl. C12Q 1/68; C12P 19/34

(52) U.S. Cl. 435/6; 435/91.2

(57) ABSTRACT

The present invention is directed to methods, compositions, kits and devices for making a nucleic acid having selected affinity to a target molecule. One embodiment of the present invention is a method of making a replicatable nucleic acid template having a selected affinity to a target. The method comprises the step of applying a selection to a first generation comprising at least one replicatable nucleic acid template as the replicatable nucleic acid template is replicated by a nucleic acid polymerase to form at least one subsequent generation comprising a replicatable nucleic acid template. The selection is based on the affinity of the replicatable nucleic template of different generations to the target. The nucleic acid polymerase introduces genetic variability between generations of the replicatable nucleic acid template to produce replicatable nucleic acid templates having different affinities to the target. The replicatable nucleic acid templates are separated on the basis of the affinity of the replicatable nucleic acid template to the target.

This application is a continuation in part application of a provisional application U.S. Ser. No. 60/091,578, filed Jul. 2, 1998, and an application filed as a Patent Cooperation Treaty (PCT) application designating the United States International Application Number PCT/US99/15030, filed Jul. 1, 1999.

### FIELD OF THE INVENTION

The present invention is directed to methods, compositions, kits and devices for making a nucleic acid molecule having selected affinity to a target molecule. One embodiment of the present invention is a method of making a replicatable nucleic acid template having a selected affinity to a target. The method comprises the step of applying a selection to a first generation comprising at least one replicatable nucleic acid template as the replicatable nucleic acid template is replicated by a nucleic acid polymerase to form at least one subsequent generation. The subsequent generation comprises at least one evolved replicatable nucleic acid template. The selection is based on the affinity of the replicatable nucleic templates of different generations to the target. The nucleic acid polymerase introduces genetic variability between generations of the replicatable nucleic acid template, directed by selection, to produce evolved replicatable nucleic acid templates having different affinities to the target. The replicatable nucleic acid templates are separated on the basis of the affinity of the replicatable nucleic acid template to the target.

### BACKGROUND OF THE INVENTION

The words and terms used in this application have definitions and meanings that one skilled in the art of molecular biology and genetics would recognize. For the purpose of convenience, the following definitions are provided.

Nucleic acid means either deoxyribonucleic acid (DNA) or ribonucleic acid (RNA), single stranded or double stranded and any chemical modifications thereof.

A replicatable nucteic acid template is a nucleic acid having a sequence of nucleotides which sequence is recognized by a polymerase to produce copies of itself. By way of example, without limitation, certain RNA bacteriophages replicate a nucleic acid, an RNA, by an RNA-dependent RNA polymerase. The enzymes Q-Beta replicase and other replicases, such as MS2 replicase (Fedoroff, N. 1975 Cold Spring Harbor Symposium 235-258), GA replicase (Yousani et al 1981 J. Biochem 89,741-750), and SP replicase (Miyare et al 1971 PNAS 68, 2022-2024) are examples of such RNA polymerases. These polymerases typically exhibit preferences for sequences having certain features, particularly their respective naturally occurring sequences or templates.

The term “generation” refers to a group of replicatable nucleic acid templates initiating or resulting from a cycle of replication. The homogeneity of the population of replicatable nucleic acid templates will depend on the fidelity of the polymerase in replicating the template. The enzyme Q-Beta replicase replicates RNA templates with low fidelity. Rather, each generation of templates will exhibit differences caused by a single nucleotide substitution, addition and deletion and the rearrangement of groups of nucleotides.

A ligand is a molecule which exhibits affinity for another molecule. A molecule of interest or target is that which one wishes to detect, identify and characterize in greater detail or create a nucleic acid ligand for. Affinity refers to the ability to bind to or form a complex with.

A kit is an assembly of parts and reagents for performing a method.

Other words and terms will be defined in the Summary of Invention and Detailed Discussion that follow.

Nucleic acids can exhibit affinity for other nucleic acids (other than Watson-Crick hybridization), proteins, carbohydrates, peptides, metabolites, and other small molecules. However, application of such nucleic acid molecules, as ligands, in the manner of antibodies, has been limited. Present techniques, for identifying nucleic acid ligands which exhibit an affinity for a chosen molecule of interest or target, are awkward. Present techniques require the synthesis of random libraries of nucleic acids. Present techniques require multiple reiterative steps.

For example, U.S. Pat. No. 5,270,163 requires the steps of contacting a library of randomized nucleic acid molecules with the target, partitioning the high affinity nucleic acid molecules from the low affinity nucleic acid molecules and amplifying the high affinity nucleic acid molecules. These steps are repeated as required. In this process, the nucleic acid molecules contacting the target is an RNA and the amplified nucleic acid is a cDNA of such RNA. The process is time consuming. Finally, one or more species of nucleic acid are identified with high affinity to the target from the many with low affinity to the target.

Embodiments of the present invention address several problems associated with identifying nucleic acid ligands. Rather than identifying one or a small number of species of nucleic acid exhibiting the highest affinity for the target, embodiments of the present invention feature the evolution of replicative nucleic acid templates which over generations exhibit a selected affinity to the target.

### SUMMARY OF THE INVENTION

The present invention is directed to methods, compositions, kits and devices for making a nucleic acid molecule having selected affinity to a target molecule. One embodiment of the present invention is a method of making a replicatable nucleic acid template having a selected affinity to a target. The method comprises the step of applying a selection to a first generation comprising at least one replicatable nucleic acid template as the replicatable nucleic acid template is replicated by a nucleic acid polymerase to form at least one subsequent generation comprising a replicatable nucleic acid template. The selection is based on the affinity of the replicatable nucleic template of different generations to the target. The nucleic acid polymerase introduces genetic variability between generations of the replicatable nucleic acid template to produce replicatable nucleic acid templates having different affinities to the target. The replicatable nucleic acid templates are separated on the basis of the affinity of the replicatable nucleic acid template to the target.

As used herein, the term “selection” refers to conditions in which variants of the replicatable nucleic acid template compete. Each variant may present a unique molecular genotype which, due to its nucleic acid composition, will represent a unique phenotype. The conditions of the selection allow variants with a particuiar phenotype to replicate at a higher rate than others. The nucleic acid polymerase introduces molecular variability with each generation, to create or evolve phenotypes and genotypes not present in the initial generation. As used herein, the term “separation” refers to the isolation of the replicatable nucleic acid template after the selection has been applied and completed.

A preferred nucleic acid is an RNA. And, a preferred RNA replicatable template is selected from the group of templates recognized by the nucleic acid polymerase. By way of example, without limitation, the enzyme Q-Beta replicase recognizes the following templates; wild-type Q-Beta phage genomic RNA, midi-varient RNA, nano-varient RNA, RQ-135 RNA and modifications of such RNAs derived from evolutionary processes.

A preferred polymerase is selected from the group of polymerases consisting of Q-Beta replicase, MS2 replicase, GA replicase, and SP replicase and modifications thereof. A particularly preferred replicase is a modified RNA polymerase exhibiting relaxed selectivity towards the template and relaxed fidelity in the replication process to create variants. Such polymerase tends to introduce and accept nucleotide substitution, deletion and insertion at a high rate. For example, a derivative of the enzyme Q-Beta replicase can be isolated which derivative is missing a protein subunit, S1, which is normally present in the wild type enzyme. The enzyme without such subunit exhibits few binding sites for the normal template.

One embodiment of the present invention features an RNA polymerase having one or more target moieties. The RNA polymerase having one or more target moieties exhibits affinity to the target to exert a selection on the RNA template as such template is replicated. A preferred RNA polymerase carries one or more target moieties covalently bound to a unit or subunit of the enzyme. By way of example, without limitation, the enzyme Q-Beta replicase has four subunits; comprising a ribosomal protein S1, EF-Ta, EF-Tu, and a phage subunit. The target is bound to or incorporated in on one or more subunits. Where the target is a protein, the nucleic acid encoding such target is cloned into the nucleic acid encoding such subunit or parts of such subunit and expressed as part of such subunit or parts thereof. In the alternative, the protein target may combined with the subunits comprising the enzyme and such subunits denatured or partially denatured in the presence of such protein target so as to fold or incorporate the protein target into the enzyme structure.

For purposes of convenience, a non-naturally occurring RNA polymerase with one or more target moieties is referred to in this application as an “evolvase”. The evolvase may have naturally occurring units and subunits to which the target is not normally bound. Replicatable RNA templates with high affinity to the target tend to be replicated with greater frequency than replicatable RNA templates with low affinity by an evolvase in the presence of suitable replication conditions. In the context of the present application, the terms “high” and “low”, as applied to affinity, refer to relative affinities. This preferential replication applies a selection for high affinity templates. The replicatable RNA templates evolve to increase the relative number of high affinity templates over successive generations.

One embodiment of the present invention features selection applied by passing a solution comprising the first and subsequent generations of the replicatable nucleic acid template through or over a fixed medium. The fixed medium has the target immobilized, such that replicatable nucleic acid templates having low affinity for the target and replicatable nucleic acid templates having high affinity for the target assume different positions in the solution as the solution passes through the fixed medium. The medium may comprise a capillary, packed column, a gel, beads, glass or plastic sheets and other particles placed in any suitable vessel.

Preferably, the fixed medium is provided with reagents and a polymerase. The reagents and polymerase are added to the fixed medium in a manner in which the replicatable nucleic acid templates may be replicated to form at least one subsequent generation. Replicatable nucleic acid templates with low affinity will assume a different position in the solution. Such low affinity replicatable nucleic acid templates will pass more quickly, be withheld less, than such replicatable nucleic acid template with high affinity. Preferably, the fixed medium allows the formation of a multitude of generations in the presence of the target. Thus, replicatable nucleic acid templates exhibiting high or low affinity, or any affinity in between, can be created. The target can be fixed by itself or attached covalently to the nucleic acid polymerase. Both the nucleic acid polymerase and the target can be immobilized.

Preferably, the solution flows through a fixed matrix. Preferably, the solution and RNA templates are influenced or acted upon by electromotive forces. These electromotive forces, in the nature of electric field electrophoresis, move the templates through the matrix.

In the alternative, selection is applied by placing the first generation and target in a solution and forming at least one subsequent generation. The method further comprises the step of forming a first fraction of the solution and a second fraction of said solution. The first fraction is substantially devoid of target. The solution of said first fraction containing replicatable nucleic acid templates having lower affinity than replicatable nucleic acid templates of the second fraction. The second fraction has the target or was closely associated with the target. This fraction contains replicatable nucleic acid templates having higher affinity than replicatable nucleic acid templates in the first fraction. Preferably, the formation of fractions is repeated or is performed in a continuous manner during the formation of multiple generations. That is, at least one fraction containing a replicatable nucleic acid template of a subsequent generation is again placed with the target in a solution. After the formation of a further subsequent generation of a replicatable nucleic acid template, a further first and second fraction is formed. Thus, replicatable nucleic acid templates exhibiting high or low affinity, or any affinity in between, can be created.

Fractions may be formed by one or more of the processes selected from the group consisting of centrifugation, dilution, filtration, electric field electrophoresis, precipitation and immobilization of the target on supports. The supports are preferably removable, such as magnetic beads, styrene beads and particles, nitrocellulose and the like.

Preferably, the selection is applied in a plurality of vessels and/or in a plurality of media to reduce selection based on the composition of the vessel.

A further embodiment of the present invention features a device for making a replicatable nucleic acid template having a selected affinity to a target. The device comprises means for applying a selection to a first generation comprising at least one replicatable nucleic acid template as the replicatable nucleic acid template is replicated by a polymerase to form at least one subsequent generation comprising a replicatable nucleic acid template. The selection is based on the affinity of the replicatable nucleic acid template of different generations to the target. The polymerase introduces molecular variability between generations of the replicatable nucleic acid template to produce replicatable nucleic acid templates having different affinities to the target. And, the device comprises means for separating the replicatable nucleic acid templates on the basis of the affinity of the replicatable nucleic acid template to the target.

Preferably, the means for applying a selection is a fixed medium. The selection is applied by passing a solution comprising the first and subsequent generations of the replicatable nucleic acid template through the fixed medium. The fixed medium has the target immobilized and capable of being bound to the replicatable nucleic acid templates. Replicatable nucleic acid templates having low affinity for the target and replicatable nucleic acid templates having high affinity for the target assume different positions in the solution as the solution passes through the fixed medium.

Preferably, the fixed medium is held in a column.

In the alternative, the fixed medium is a matrix upon which an electromotive force is placed. Or, the means for applying a selection is a non-naturally occurring polymerase having a target moiety, a evolvase.

In a further embodiment of the present invention, the means for applying a selective vector comprises a plurality of vessels constructed and arranged to receive fractions of solutions. The fractions contain replicatable nucleic acid templates exhibiting different affinities to the target. The replicatable nucleic acid templates exhibiting a trend toward the desired affinity are allowed to form at least one further generation.

A further embodiment of the present invention features a kit for making a replicatable nucleic acid template having a selected affinity to a target. The kit comprises means for applying a selection to a first generation comprising at least one replicatable nucleic acid template as the replicatable nucleic acid template is replicated by a polymerase to form at least one subsequent generation comprising a replicatable nucleic acid template. The selection is based on the affinity of the replicatable nucleic acid template of different generations to the target. The polymerase introduces genetic variability between generations of the replicatable nucleic acid template to produce replicatable nucleic acid templates having different affinities to the target. And, the kit comprises means for separating the replicatable nucleic acid templates on the basis of the affinity of the replicatable nucleic acid template to the target.

A further embodiment of the present invention comprises an evolvase.

These and other features and advantages will be apparent to individuals skilled in the art in light of the drawings and the detailed description of the invention that follow.

### BRIEF DESCRIPTION OF THE DRAWINGS

FIG. 1 depicts the nucleic acid sequence of one replicatable nucleic acid template for the enzyme Q-Beta replicase;

FIG. 2 depicts the enzyme having features of the present invention;

FIG. 3 depicts in schematic form a device embodying features of the present invention;

FIG. 4 depicts in schematic form a further device embodying features of the present invention;

FIG. 5 depicts in schematic form a further device embodying features of the present invention; and,

FIG. 6 depicts a kit having features of the present invention.

### DETAILED DESCRIPTION OF THE DRAWINGS

The present invention is directed to methods, kits and devices for making a nucleic acid having selected affinity to a target molecule. The invention and the figures will be described in detail with respect to a method of making a replicatable nucleic acid template having a selected affinity to a target. The method comprises the step of applying a selection to a first generation comprising at least one replicatable nucleic acid template as the replicatable nucleic acid template is replicated by a nucleic acid polymerase to form at least one subsequent generation comprising a replicatable nucleic acid template. The selection is based on the affinity of the replicatable nucleic template of different generations to the target. The nucleic acid polymerase introduces genetic variability between generations of the replicatable nucleic acid template to produce replicatable nucleic acid templates having different affinities to the target. The replicatable nucleic acid templates are separated on the basis of the affinity of the replicatable nucleic acid template to the target.

A preferred nucleic acid is an RNA. One replicatable RNA template is midi-variant RNA, the sequence of which is set forth below in Seq ID No 1:

Seq ID No.1

5′-GGGGACCCCC CCGGAAGGGG GGGACGAGGU GCGGGCACCU
CGUACGGGAG UUCGACCGUG ACGCUCUAGA GAUCUAGAGC
ACGGGCUAGC GCUUUCGCGC UCUCCCAGGU GACGCCUCGU
GAAGAGGCGC GACCUUCGUG CGUUUCGGUG ACGCACGAGA
ACCGCCACGC UGCUUCGCAG CGUGGCUCCU UCGCGCAGCC
CGCUGCGCGA GGUGACCCCC GAAGGGGGGU UCCC-3′

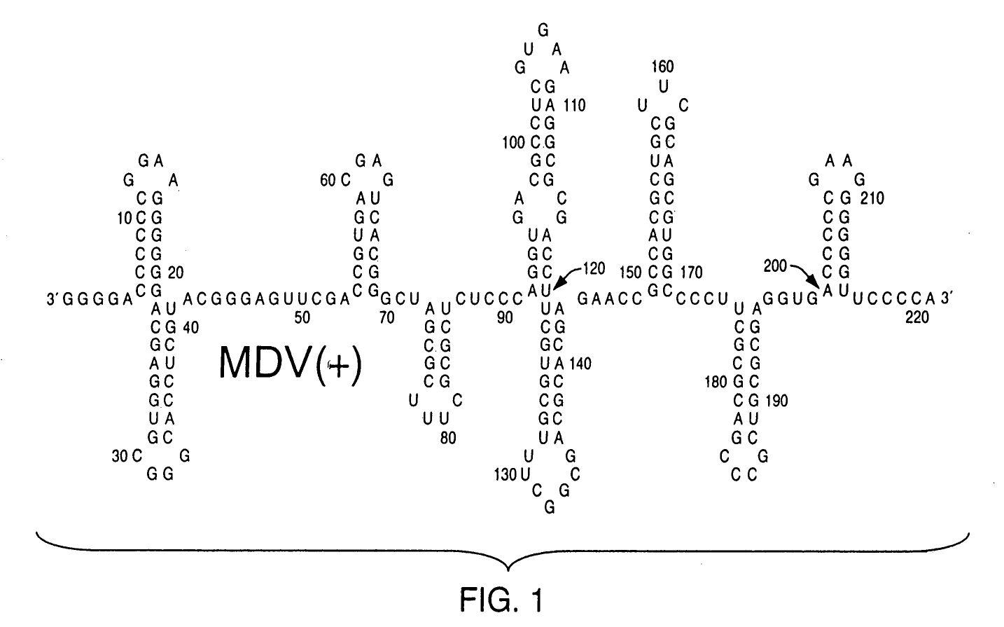

FIG. 1 depicts in schematic form naturally occurring MDV(+) RNA. Nishiharra et al (1983). Other replicatable RNA templates may also be used depending on the choice of polymerase. With respect to the enzyme Q-Beta replicase, wild type replicatable RNA, Q-Beta bacteriophage genomic RNA, may be preferred.

A preferred polymerase is selected from the group of polymerases consisting of Q-Beta replicase, MS2, GA, and SP and modifications thereof. A particularly preferred replicase is a modified RNA polymerase exhibiting relaxed selectivity towards the template and relaxed fidelity in the replication process to create molecular variants. A relaxed fidelity RNA replicase is described by Yoshio Inokuchi and Mayayuki Kajitani, “Deletion Analysis of QB Replicase,” J. Bio Chem., vol. 272, no. 24, 15339-15345 (1997). The variants of the replicatable nucleic acid template compete for replication by the polymerase, with each other and each prior generation of replicable nucleic acid templates.

One preferred enzyme, referred to herein as an evolvase, is represented by the formula below:
C-T,
wherein C represents a catalytic entity and T represents a target. The target is covalently bound to the catalytic entity or bound by affinity. Methods and compositions for affinity coupling, such as biotin and strepavidin, or immunological reactions are well-known. Where the target is a protein, the chemistry to covalently bind a protein to the catalytic entity is well-known. A preferred catalytic entity is selected from the group of polymerases consisting of Q-Beta replicase, MS2, GA, and SP and modifications thereof. A particularly preferred replicase is a modified RNA polymerase exhibiting relaxed fidelity in the replication process to create molecular variants. For example, a derivative of the enzyme Q-Beta replicase can be isolated which derivative is missing a protein subunit, S1, which is normally present in the wild type enzyme. The enzyme without such subunit exhibits few binding sites for the normal template. This particular derivative of the enzyme Q-Beta replicase is found as a fraction of isolates from bacterial derived enzyme.

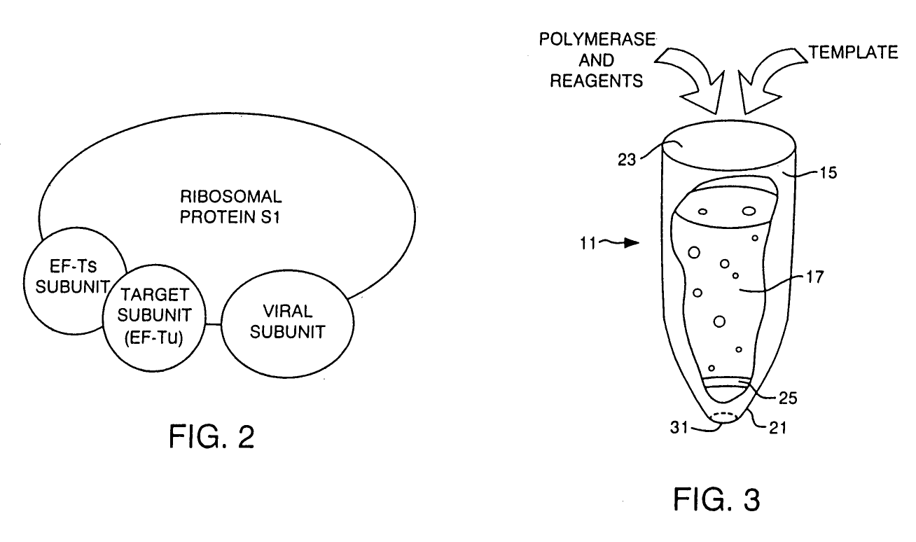

FIG. 2 depicts an evolvase derived from Q-Beta replicase. As depicted, the evolvase comprises a ribosomal protein S1, EF-Ts, a phage subunit and in the place of a subunit, EF-Tu, a target protein subunit. A target subunit protein is formed by cloning sequences for the target into sequences for the subunit EF-Tu.

In the alternative, the sequences encoding the target can be cloned into sequences for the other subunits. The cloned sequences are expressed and the evolvase isolated.

In the further alternative, a polymerase is chemically altered to covalently attach the target to an existing enzyme through protein chemistry. The formula above is not intended to reflect a strict stoichiometric relationship between the catalytic entity and the target. Indeed, a plurality of target entities may be combined with each catalytic entity.

The evolvase is placed in contact with a nucleic acid template. The template is replicated by imposing reaction conditions. In the evolvase based on the enzyme Q-Beta replicase, the reaction conditions would be identical or similar to those for such enzyme. Typical reagents and conditions comprise 173 mM Tris-HCl, pH 7.5, 27 mM MgCl₂, and 0.77 mM each ATP, GTP, CTP, and UTP. Upon imposing reaction conditions, successive generations of the template are formed. Molecular variability is introduced in the successive generations by the evolvase. Those templates with the greatest affinity to the target will form the dominant species. These dominant species may be isolated or serially diluted and reapplied to the evolvase for further evolution. The early generations will exhibit low affinity to the target. The latter generations will exhibit high affinity to the target.

In the alternative, a preferred selection is applied by passing a solution comprising the first and subsequent generations of the replicatable nucleic acid template through or over a fixed medium. The fixed medium has the target immobilized, such that replicatable nucleic acid templates having low affinity for the target and replicatable nucleic acid templates having high affinity for the target assume different positions in the solution as the solution passes through or over the fixed medium. The medium may comprise a capillary, packed column, a gel, beads, glass or plastic sheets, and other particles placed in any suitable vessel.

Turning now to FIG. 3, a device for making a nucleic acid having selected affinity to a target molecule is illustrated in partial cut-away form. The device, generally designated by the numeral 11, comprises the following major elements, a housing 15 and a support 17. The housing 11 has a cylindrical shape having two ends 21 and 23. Housing 15 is preferably made of plastic or glass. As illustrated, one end 21 has a taper for retaining support 17. To further retain the support 17, the device 11 has a frit 25 retained in housing 15 at the tapered end 21. Tapered end 21 has an opening 31 to allow solutions to exit from the housing 15. The opposing end 23 is adapted to receive replicatable nucleic acid templates, a polymerase and reagents for replication.

The support 17, as illustrated, comprises a matrix such as Activated CH Sepharose 4B (Pharmacia LKB). The target, in the case of proteins, is attached to the matrix according to standard procedures. The activated groups remaining on the matrix are blocked with Tris.

In operation, the housing 15 is loaded with the support 17 to which the target is affixed. The support 17, a fixed medium, is provided with a replicatable nucleic acid template. A preferred template is Q-Beta bacteriophage genomic RNA. The template is preferably allowed to migrate into the support 17 matrix.

Reagents and a polymerase are added to the support 17 matrix. The reagents and polymerase are added to the support 17 in a manner in which the replicatable nucleic acid templates may be replicated to form at least one subsequent generation. Where the polymerase is Q-Beta replicase, typically, the reagents comprise 173 mM Tris-HCl, pH 7.5, 27 mM MgCl₂, and 0.77 mM each ATP, GTP, CTP, and UTP. Where the housing 15 is sized to correspond approximately with the size of an Epindorf tube, approximately 1.2 μg Q-Beta replicase is added. As the reaction proceeds, the solution is drawn through the housing 15 through the support 17 by means of vacumn at end 21, pressure at end 23 or centripetal force by means of a centrifuge (not shown). As the reaction proceeds, new generations of the template are formed. Due to the lack of template specificity and the lack of replication fidelity of the enzyme, the templates will evolve, losing or gaining or substituting single nucleotides or nucleic acid sequences, in response to the selection.

Replicatable nucleic acid templates with low affinity will assume a different position in the solution. Such low affinity replicatable nucleic acid templates will pass more quickly, be withheld less, than such replicatable nucleic acid template with high affinity. Templates exhibiting high affinity will form more generations being be held in the support 15 for a longer period. Thus, replicatable nucleic acid templates exhibiting high or low affinity, or any affinity in between, can be created.

Templates with high affinity to the target can be subjected to further selection or modification to evolve such templates for low affinity to the support 17. That is, the device 11 is used with a support 17 which support 17 does not have target. Templates which pass through the support 17 first have low affinity for the support 17.

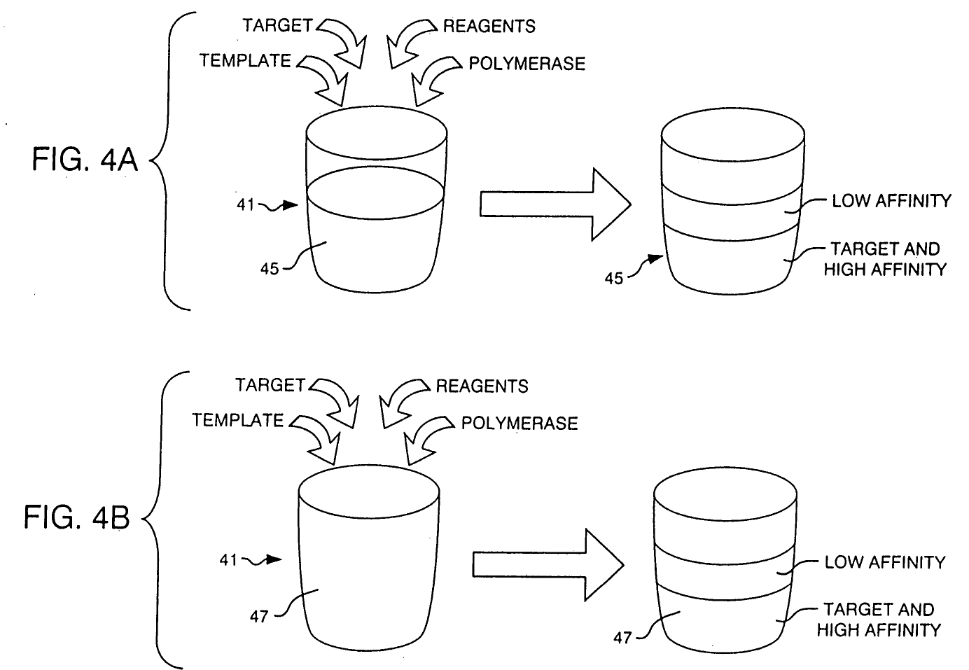

FIGS. 4a and b depicts a further embodiment of the present invention. Turning first to FIG. 4a, a device, generally designated by the numeral 41 for making a nucleic acid having selected affinity to a target molecule is illustrated. The device 41 comprises a first vessel 45. First vessel 45 receives a replicatable nucleic acid template, target, reagents and polymerase. Upon imposing reaction conditions, at least one subsequent generation of template is formed. The double arrow represents imposing fraction forming conditions on the target. These conditions may be imposed by chemical or physical means, such as the addition of salt, change in pH, of centrifugation. The imposition of fraction forming conditions forms a first fraction of the solution and a second fraction of said solution. The first fraction is substantially devoid of target. The solution of said first fraction contains replicatable nucleic acid templates having lower affinity than replicatable nucleic acid templates of the second fraction. The second fraction has the target or was closely associated with the target. This fraction contains replicatable nucleic acid templates having higher affinity than replicatable nucleic acid templates in the first fraction.

Turning now to FIG. 4b, a second vessel 47 receives a replicatable nucleic acid template, target, reagents and polymerase. The replicatable nucleic acid template is selected for high or low affinity. Upon imposing reaction conditions, at least one subsequent generation of template is formed. The double arrow represents imposing fraction forming conditions on the target. These conditions may be imposed by chemical or physical means, as discussed with respect to FIG. 4a. The imposition of fraction forming conditions forms a first fraction of the solution and a second fraction of the solution. The first fraction is substantially devoid of target. The solution of the first fraction contains replicatable nucleic acid templates having lower affinity than replicatable nucleic acid templates of the second fraction. The second fraction has the target or was closely associated with the target. This fraction contains replicatable nucleic acid templates having higher affinity than replicatable nucleic acid templates in the first. Preferably, the formation of fractions is repeated or is performed in a continuous manner during the formation of multiple generations. Preferably, the movement of selected fractions, the imposition of fraction forming conditions, the addition of target, reagents, and polymerase is automated. Thus, replicatable nucleic acid templates exhibiting high or low affinity, or any affinity in between, can be created.

Preferably, the selection is applied in a plurality of vessels and/or in a plurality of media to reduce selection based on the composition of the vessel.

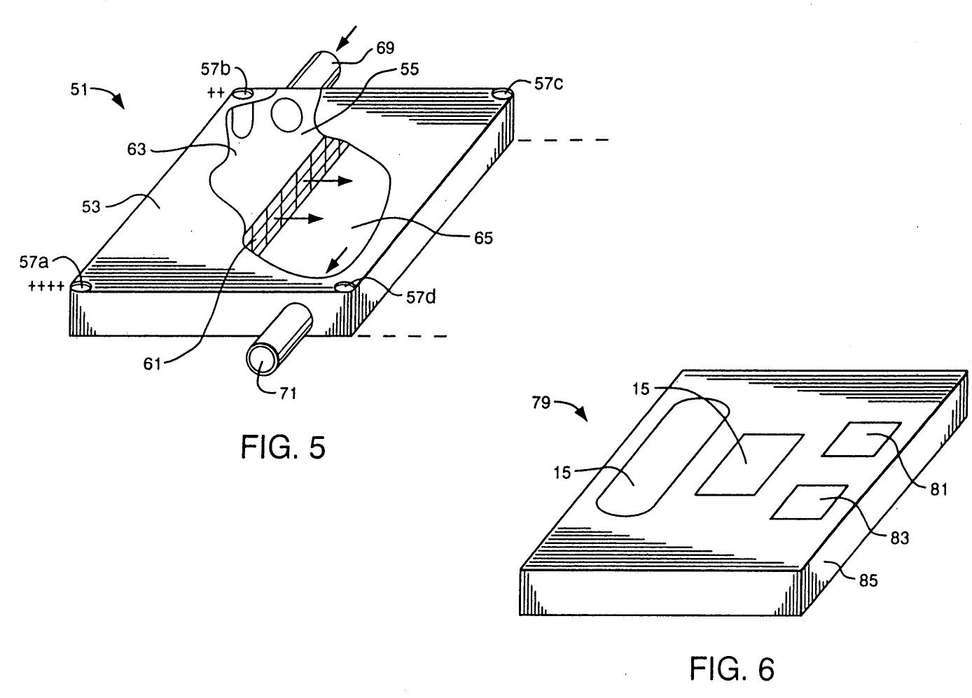

Turning now to FIG. 5, a further embodiment of a device, generally designated by the numeral 51, embodying features of the present invention, is illustrated in partial cut-away. Device 51 comprises the following major elements: a housing 53, a matrix 55, and electrodes 57a-d.

Housing 53 defines a containment vessel for holding matrix 55 and a solution containing replicatable nucleic acid templates and reagents. A permeable divider 61 divides the housing into a first compartment 63 and a second compartment 65. Matrix 55 is held in a first compartment 63 or comprises one or more the walls of first compartment 63. Such walls may be pitted to form micro volumes. Second compartment 65 provides a fluid passage for fluids exiting matrix 55.

Housing 53 has an inlet 69 for receiving a solution containing one or more nucleic acid templates and other reagents and directing such solution to matrix 55. Inlet 69 is, preferably, in fluid communication with a continuous source (not shown) of templates and reagents and controlled by suitable valves and automated controls (not shown). Housing 53 has an outlet 71 for discharging fluid.

Matrix 55 comprises a packing or support material to which target or an evolvase (not shown) is immobilized. Matrix 55 allows solution to flow carrying replicatable nucleic acid templates. Under condition for replication, the templates evolve with templates with the greatest affinity for the target being retained in the matrix 55.

Electrodes 57a-d provide electromotive force to compel templates exhibiting weak affinity for the target to exit the matrix 55. Thus, electrodes 57b and c exert a weak electromotive force on templates entering the matrix 55 and electrodes 57a and d exert a more powerful electromotive force on templates exiting the matrix 55 toward the outlet 71.

In operation, replicatable nucleic acid templates placed in the matrix 55 at the inlet 69 with a polymerase and suitable reagents. Templates are created with varying degrees of affinity to the target. Templates exhibiting weak affinity to the target are withdrawn from the matrix 55 by electromotive force created by electrodes 57a-d and conveyed from the housing 51 via second compartment 65 and outlet 71.

Templates with higher affinity to the target are retained in the matrix 55 and undergo further replication and evolution. Provided with reagents, the templates exiting outlet 71 will become constant or begin to taper off as templates evolve with such high affinity that such templates are bound to target and are unavailable to replicate. Such templates can be isolated from the target by denaturing the matrix bound target.

FIG. 6 depicts a kit, generally designated by the numeral 79, for making a replicatable nucleic acid template having a selected affinity to a target. The kit comprises a housing 15 and support 17 for applying a selection to a first generation comprising at least one replicatable nucleic acid template as the replicatable nucleic acid template is replicated by a polymerase to form at least one subsequent generation comprising a replicatable nucleic acid template. The selection is based on the affinity of the replicatable nucleic acid template of different generations to the target. The polymerase introduces genetic variability between generations of the replicatable nucleic acid template to produce replicatable nucleic acid templates having different affinities to the target. The housing 15 and support also facilitate separating the replicatable nucleic acid templates on the basis of the affinity of the replicatable nucleic acid template to the target. Preferably, the support 17 has target immobilized on it surface, or is functionalized to allow an individual skilled in the art to place the target of his/her choice on the support. The kit further comprises suitable reagents and polymerases held in appropriate vessels 81-83 (additional vessels may be required). The kit, preferably, is held in suitable packaging, such as a box 85, and provided with instructions for use.

A preferred kit comprises an evolvase.

While the preferred embodiments of the present invention have been illustrated and described, it is understood that the present invention is capable of variation and modification and therefore, should not be limited to the precise details set forth, but should include such changes and alterations that fall within the purveiw of the following claims.

1. A method of making a replicatable RNA template having a selected affinity to a target, comprising the steps of:
a.) applying a selection to a first generation comprising at least one replicatable RNA template as said replicatable RNA template is replicated by a RNA polymerase to form at least one subsequent generation comprising a replicatable RNA template, said selection based on the affinity of said replicatable RNA template of different generations to said target, said RNA polymerase introducing genetic variability between generations of said replicatable RNA template to produce replicatable RNA templates having different affinities to said target; and
b.) separating the replicatable RNA templates on the basis of the affinity of said replicatable RNA template to the target.

2. The method of claim 1 wherein said selection is applied by passing a solution comprising said first and subsequent generations of said replicatable RNA template through or over a fixed medium, said fixed medium having said target immobilized, such that replicatable RNA templates having low affinity for the target and replicatable RNA templates having high affinity for the target assume different positions in said solution as said solution passes through said fixed medium.

3. The method of claim 2 wherein said fixed medium is provided with reagents and an RNA polymerase.

4. The method of claim 3 wherein said RNA polymerase is selected from the group of polymerases consisting of Q-Beta replicase, MS2 replicase, GA replicase, and SP replicase and modifications thereof.

5. The method of claim 4 wherein said RNA polymerase is non-selective with respect to template.

6. The method of claim 1 wherein said selection is applied by placing said first generation and target in a solution, and after at least one subsequent generation has been formed, forming a first fraction of said solution and a second fraction of said solution, said first fraction substantially devoid of target, said solution of said first fraction containing replicatable RNA templates having lower affinity than replicatable RNA templates in said second fraction, said second fraction having said target containing replicatable RNA templates having higher affinity than replicatable RNA templates in said first fraction.

7. The method of claim 6 wherein said separation is performed by one or more of the processes selected from the group consisting of centrifugation, filtration, electric field electrophoresis, and precipitation.

8. The method of claim 6 wherein at least one fraction is provided with additional reagents and RNA polymerase in the presence of target.

9. The method of claim 6 wherein said selection is applied in a plurality of vessels containing said solution to reduce selection based on the composition of the vessel.

10. The method of claim 1 wherein said selection is applied by an enzyme having a catalytic entity and a target entity.

11. The method of claim 10 wherein said catalytic entity is derived from one of the RNA dependent RNA polymerases selected from the group consisting of Q-Beta replicase, MS2 replicase, GA replicase, and SP replicase and modifications thereof.

12. The method of claim 10 wherein said target is incorporated into one or more subunits of the polymerase.

13. The method of claim 10 wherein said target is covalently bonded to said catalytic entity.

14. A device for making a replicatable RNA template having a selected affinity to a target, comprising:
a.) means for applying a selection to a first generation comprising at least one replicatable RNA template as said replicatable RNA template is replicated by a RNA polymerase to form at least one subsequent generation comprising a replicatable RNA template, said selection based on the affinity of said replicatable RNA template of different generations to said target, said RNA polymerase introducing genetic variability between generations of said replicatable RNA template to produce replicatable RNA templates having different affinities to said target; and
b.) means for separating the replicatable RNA templates on the basis of the affinity of said replicatable RNA template to the target.

15. The device of claim 14 wherein said means for applying a selection is a fixed medium said selection applied by passing a solution comprising said first and subsequent generations of said replicatable RNA template through said fixed medium, said fixed medium having said target immobilized, such that replicatable RNA templates having low affinity for the target and replicatable RNA templates having high affinity for the target assume different positions in said solution as said solution passes through said fixed medium.

16. The device of claim 11 wherein said fixed medium is held in a column.

17. A kit for making a replicatable RNA template having a selected affinity to a target, comprising:
a.) means for applying a selection to a first generation comprising at least one replicatable RNA template as said replicatable RNA template is replicated by a RNA polymerase to form at least one subsequent generation comprising a replicatable RNA template, said selection based on the affinity of said replicatable RNA template of different generations to said target, said RNA polymerase introducing genetic variability between generations of said replicatable RNA template to produce replicatable RNA templates having different affinities to said target; and
b.) means for separating the replicatable RNA templates on the basis of the affinity of said replicatable RNA template to the target.

18. The Kit of claim 17 wherein said RNA polymerase is evolvase.
